(19) United States
(12) Patent Application Publication
(10) Pub. No.: US 2003/0229903 A1
(43) Pub. Date: Dec. 11, 2003

(54) Novel system for the evaluation of the activity and/or specificity of a viral component

(76) Inventors: Matthias Renner, Wien (AT); Walter Gunzburg, Modling (AT)

Correspondence Address: BAKER & BOTTS, 30 ROCKEFELLER PLAZA, NEW YORK, NY, 10112

(21) Appl. No.: 10/410,469

(22) Filed: Apr. 9, 2003

Related U.S. Application Data
(63) Continuation of application No. PCT/EP01/11626, filed on Oct. 8, 2001.

(30) Foreign Application Priority Data
Oct. 10, 2000 (DK) PA 2000 01519

(51) Int. Cl. C12Q 1/70; A01K 67/00

(52) U.S. Cl. 800/3; 800/8; 435/5

(57) ABSTRACT

The present invewntion relates to a method for the evaluation of the activity and/or specificity of a regulatory sequence or of a viral component, wherein a viral vector is introduced into a cell of a transgenic non-human animal comprising in its genome one or more viral sequences. However, said transgenic animal is deficient in at least one viral sequence required for generation of the virus. This viral sequence is included in the viral vector introduced into the cell of the transgenic animal, thereby allowing reconstitution of viral particle generation in the transgenic animal. After maintaining the transgenic animal under suitable conditions allowing the production of viral particles, the cells in which viral particles are produced can be detected and evaluated, respectively. The method according to the present invention can also be adapted to evaluate the distribution of a receptor for a ligand in an animal. The present invention also provides a transgenic non-human animal applicable for use in the method according to the present invention.

The present invention relates to a method for the evaluation of the activity and/or specificity of a regulatory sequence or of a viral component, wherein a viral vector is introduced into a cell of a transgenic non-human animal comprising in its genome one or more viral sequences. However, said transgenic animal is deficient in at least one viral sequence required for generation of the virus. This viral sequence is included in the viral vector introduced into the cell of the transgenic animal, thereby allowing reconstitution of viral particle generation in the transgenic animal. After maintaining the transgenic animal under suitable conditions allowing the production of viral particles, the cells in which viral particles are produced can be detected and evaluated, respectively. The method of the present invention can also be adapted to evaluate the distribution of a receptor for a ligand in an animal. The present invention also provides a transgenic non-human animal applicable for use in the method according to the present invention.

### BACKGROUND OF THE INVENTION

The use of viral vectors, especially of retroviral vectors, for gene therapy has received much attention and currently is the method of choice for the transferral of therapeutic genes in a variety of approved protocols both in the USA and in Europe (Kotani, H. et al. 1994, Human Gene Therapy 5: 19-28). However most of these protocols require that the infection of target cells with the retroviral vector carrying the therapeutic gene occurs in vitro, and successfully infected cells are then returned to the affected individual (Rosenberg et al. 1992, Human Gene Therapy 3: 75-90; Anderson, W. F. 1992, Science 256: 808-813). Such ex vivo gene therapy protocols are ideal for correction of medical conditions in which the target cell population can be easily isolated (e.g. lymphocytes). Additionally the ex vivo infection of target cells allows the administration of large quantities of concentrated virus which can be rigorously safety tested before use.

Unfortunately, only a fraction of the possible applications for gene therapy involve target cells that can be easily isolated, cultured and then reintroduced. Additionally, the complex technology and associated high costs of ex vivo gene therapy effectively preclude its disseminated use world-wide. Future facile and cost-effective gene therapy will require an in vivo approach in which the viral vector, or cells producing the viral vector, are directly administered to the patient in the form of an injection or simple implantation of retroviral vector producing cells.

This kind of in vivo approach, of course, introduces a variety of new problems. First of all, and above all, safety considerations have to be addressed. Virus will be produced, possibly from an implantation of virus producing cells, and there will be no opportunity to precheck the produced virus. It is important to be aware of the finite risk involved in the use of such systems, as well as trying to produce new systems that minimize this risk.

For therapeutic purposes the retroviral vector itself is normally a modified vector in which the genes encoding retroviral proteins have been replaced by therapeutic genes and marker genes to be transferred to the target cell. Since the replacement of the genes encoding the retroviral proteins effectively cripples the virus, the virus is replication deficient and must be rescued by a component which provides the missing viral proteins to the modified retrovirus. This component is regularly a cell line that produces large quantities of the viral proteins, however lacks the ability to produce replication competent virus. This cell line is known as the packaging cell line and consists of a cell line transfected with a second plasmid carrying the genes enabling the modified retroviral vector to be packaged.

To generate the packaged vector, the vector plasmid is transfected into the packaging cell line. Under these conditions the modified retroviral genome including the inserted therapeutic and marker genes is transcribed from the vector plasmid and packaged into the modified retroviral particles (recombinant viral particles). This recombinant virus is then used to infect target cells in which the vector genome and any carried marker or therapeutic genes becomes integrated into the target cell's DNA. A cell infected with such a recombinant viral particle cannot produce new viruses since not all retroviral proteins are present in these cells. However, the DNA of the vector carrying the therapeutic and marker genes is integrated in the cell's DNA and can now be expressed in the infected cell.

A further consideration when considering the use of in vivo gene therapy, both from a safety point of view and from a purely practical point of view, is the targeting of retroviral vectors. It is clear that therapeutic genes carried by vectors should not be indiscriminately expressed in all tissues and cells, but rather only in the requisite target cell. This is especially important if the genes to be transferred are toxin or suicide genes aimed at ablating specific tumour cells. Ablation of other, nontarget cells would obviously be very undesirable.

A number of retroviral vector systems have been described that should allow targeting of the carried therapeutic genes (Salmons, B. and Günzburg, W. H. 1993, Human Gene Therapy 4: 129-141). Most of these approaches involve either limiting the infection event to predefined cell types or, more often, using heterologous regulatory sequence to direct expression of linked heterologous therapeutic or marker genes to specific cell types. However, the eucaryotic regulation of the expression of a gene is a very complex process which is still not fully understood. The DNA sequences that control the expression of a gene are called “regulatory sequences”. These sequences comprise generally a promoter, where the general transcription factors and the polymerase assemble, as well as sequences to which gene regulatory proteins bind to control the rate of these assembly processes at the promoter. The promoter is normally found immediately 5′ to the gene. The sequences, to which the regulatory proteins bind, thereby acitivating or suppressing the transcription of the gene, are located further upstream of the gene and are called “enhancer” and/or “suppressor”. In higher eucaryotes it is not unusual to find the regulatory sequences of a gene dotted over distances as great as 50,000 nucleotide pairs, although much of this DNA serves as “spacer” sequence and is not recognized by gene regulatory proteins.

If a regulatory sequence is used in a vector to drive the expression of a gene, only short stretches of nucleic acid sequences can be introduced into the vector due to the limited capacity of retroviral vectors. Accordingly, only parts of the regulatory sequence can be used to drive the expression of the marker or therapeutic gene in a vector. In order to obtain a regulatory sequence usable for a retroviral vector e.g. for gene therapy, the regions within said regulatory sequence have to be identified which are required for the general function of the regulatory sequence and especially for tissue specificity. The knowledge about the exact sequence motifs defining the activity and tissue specificity of a regulatory sequence would allow the design of an optimum promoter for a specific application e.g. in gene therapy.

However, only limited information is available about the essential regions of a regulatory region. Computer models are available which predict functional domains based on the nucleic acid sequence. However, that's only a prediction. The real functional domains may be different, or simply the motives are still unknown. Other methods are based on protein binding assays such as footprinting, wherein a functional sequence within a regulatory region is indicated by any protein binding to it. However, the method does not allow to conclude from the protein binding to the function of these sequence motifs, if they are enhancers, silencers, essential or alternative. The most useful method available at the moment for the analysis of regulatory sequence is a reporter gene assay. According to this method, the original or a mutated version of the promoter is fused upstream of a reporter gene, i.e. a gene encoding an easily measurable protein or protein activity so that transcripts initiating at the promoter proceed through the reporter gene. Subsequently, the construct is transduced into living cells of a cell culture system and the amount and/or activity of reporter protein and thereby the promoter activity is determined. However, cells in a cell culture system often poorly correspond to the natural cells. Most cells are immortalized, i.e. can be cultured over an unlimited number of passages. However, with each passage the characteristics of the cells change and they become less typical for the cells of the organ they are derived from. Even if the cells are freshly isolated from an organ, i.e. correspond as closely as possible to the cells of the organ, they often do not show any more the original characteristics for this cell type since they lack the natural system such as the surrounding cells and the natural extracellular matrix. Consequently, the promoter in the reporter gene construct does not react the same way as in the natural system. Hence, even if in the natural system the promoter would be active in the presence of specific factors, the reporter gene construct tested in the cell culture system in some cases wouldn't be active and the other way round. Hence, the results of reporter gene assays in vitro are often misleading.

Consequently, to obtain definitive results, in vivo studies in experimental animals are required. If the activity of the promoter in different cell types is analyzed, animals transgenic for the regulatory sequence of interest are used. Transgenic animals are produced e.g. by microinjection, i.e. by injection of a heterologous gene construct into the nuclei of 1-cell stage embryos or by infection of preimplantation embryos with retroviral vector DNA (Gordon J. et al. 1980, Proc. Natl. Acad. Sci, USA 77: 7380-7384, Jaenisch R. et al. 1983, Cell 32: 209-216). The resulting embryos can further develop to full term after the transfer into the oviducts/uteri of recipient foster mothers. Some of the resulting adult animals have the heterologous DNA integrated into their genome. If the germ line cells (eggs or sperm) are modified all animals developing from said germ cells contain in all cells of the body including the germ cells the foreign nucleic acid sequence and pass it on to its progeny. Such in their germ line modified animals are called “transgenic” and the foreign DNA for which they are transgenic is called “transgene”. Although transgenic animals are produced routinely mostly by specialized laboratories, the process is laborious, time-consuming, expensive, and sometimes has to be repeated since the first attempt may even fail to produce a single transgenic animal. Accordingly, promoter analyses based on the production of transgenic animals require a high effort and therefore can only be performed for one or a very limited number of promoter variants.

To circumvent the production of transgenic animals for the analysis of promoter activity, another method was tested: Animals were infected with retroviral vectors carrying a promoter-reporter-gene construct. According to the retroviral life cycle, the retroviral vector enters a cell, is integrated into the genome of this cell and, subsequently, the reporter gene is expressed in those cells in which the regulatory sequence is active. However, in vivo transduction efficiency of safe retroviral vectors which are used for experimental and therapeutic purposes is very low. Hence, only sporadically cells are transduced resulting in single cells expressing the reporter gene surrounded by a large majority of cells which are not transduced and hence do not express the reporter gene. However, due to the low number of infected cells it is very difficult if not completely impossible to detect reporter gene activity. Especially it is difficult to distinguish between the situation when the regulatory sequence of interest is not active at all in a certain tissue and when the expression level is low and therefore can not be detected. Even if the expression of the reporter gene is rather high the infected cells might be overlooked, especially in cases in which only very few cells are infected. Accordingly, regulatory sequences can not be analyzed by introducing a promoter-reporter-gene construct into a retroviral vector.

A further problem in targeting the delivery of genes to predefined cell types may also be the infection spectrum of enveloped viruses. The infection spectrum of enveloped viruses is determined by the interaction between viral surface proteins encoded by a viral surface protein gene (“env-gene”), and host cell membrane proteins which act as receptors. Vectors derived from viruses will deliver genes in the same cell types as the original virus does, unless the infection spectrum of the vector virus is modified. One way to combine the ability of viruses to target particular cell types at the level of infection is to create “pseudotyped” viral particles which may carry the core and genetic information of one virus, and in addition the surface protein of an other virus comprising a limited infection spectrum.

It has also been described that the modification of the viral surface proteins may result in a targeted infection of specific cells: Ecotropic retrovirus do not infect human cells since human cells do not express a receptor which functionally interacts with the binding sequences on the viral surface protein. The insertion of receptor ligands (Kasahara et al. (1994), Science 266, 1373-1376), functional fragments thereof (Valsesia-Wittmann et al. (1996), J. Virol. 70, 2059-2064) or of the binding regions of monolclonal antibodies (Russell et al. (1993), Nucl. Acids Res. 21, 1081-1085) into the binding region of the viral surface protein resulted in a specific targeting of the modified retroviruses to specific cell types expressing the corresponding receptors.

From a safety point of view it would be very desirable to predetermine the infection spectrum of viruses to ensure that vector viruses carrying, e.g., a toxic therapeutic gene will only infect the requisite target cell. Moreover, it would be desirable to have a system allowing the determination of the tissue and cell type distribution of a cellular receptor or a cellular ligand.

### OBJECT OF THE INVENTION

It is an object of the present invention to provide an improved system and a fast and inexpensive method for the evaluation of the activity and/or specificity of a regulatory sequence or of viral components which are tissue and/or tumor specific and which maintain their activity and specificity in vivo even when included into heterologous systems, as, e.g., genetic constructs. Examples for regulatory sequences are promotors and enhancers. Prefered viral components are viral surface proteins. It is a further object of the present invention to provide a system that allows to determine the tissue and cell type distribution of a cellular receptor or a cellular ligand. Said methods should be especially applicable for the evaluation of viral components used in viral vectors for the expression of therapeutic and marker genes in gene therapy.

### DETAILED DESCRIPTION OF THE INVENTION

To achieve the foregoing and other objects, the present invention provides a method, wherein a viral vector is introduced into a cell of a transgenic non-human animal, preferably a mammal, more preferably a rodent and most preferably a mouse, comprising in its genome one or more viral sequences. However, said transgenic animal is deficient in at least one viral sequence required for generation of the virus. These viral sequences are included in the viral vector introduced into the cell of the transgenic animal, thereby allowing reconstitution of viral particle generation in the transgenic animal. After maintaining the transgenic animal under suitable conditions allowing the production of viral particles, the cells in which viral particles are produced can be detected and evaluated, respectively.

The method according to the present invention is especially useful for the evaluation of the activity and/or specificity of a regulatory sequence. In this case expression and/or translation of the one or more viral sequences included in the vector are regulated by the regulatory sequence to be analysed. When cells of the transgenic animal are transfected with the viral vector or infected with a viral particle comprising the vector, at first only a small number of cells are transduced and infected, respectively. Generally, all cells of the transgenic animal should be transducable and/or infectable, but expression and/or translation of the sequence(s) and gene(s), respectively, included in the vector will only occur in cells in which the regulatory sequence is active. Thus, in case that, e.g., a promoter is used which should be cell type or tissue specific it can be evaluated if the promoter indeed fulfills this expectation: Only in these cells of the transgenic animal expression and translation of the viral sequences included in the vector and required for viral particle generation should take place.

Additionally, the cells of the transgenic animal provide the further viral proteins the viral vector does not encode and, thus, have a function similar to that of a packaging cell line. Preferably, the viral proteins encoded by the viral sequence(s) included in the genome of the transgenic animal are produced permanently. These viral sequences are accordingly and preferably regulated by an ubiquitous, constitutively active regulatory sequence. Most preferably the MLV-promoter is used. However, the viral genome is packaged into viral particles only in those cells, in which the regulatory sequence included in the vector and regulating expression or translation of the gene(s) further required for generation of viral particles is active. Subsequently, only these cells of the transgenic animal release numerous, newly produced viral particles. The viral particles, then, infect other cells and the process is repeated. Hence, a kind of “snowball effect” is launched, resulting in producing a large number of viral particles infecting new cells. However, again only those cells in which the viral vector promoter is active release further viral particles.

The regulatory sequence included in the vector preferably regulates expression and/or translation of the gene encoding the envelope protein which is most preferably an amphotropic viral envelope protein and most preferably VSV-g or GALV. Generally, “amphotropic” proteins comprise proteins being cell type unspecific, i.e. these proteins may be active in different cell types and even in different species. Accordingly, viral particles comprising an amphotropic envelope protein can infect different cell types of different tissues in different host species. According to a preferred embodiment of the method provided by the present invention, the gene encoding an amphotropic envelope protein is included in the viral vector and is regulated by the regulatory sequence which should only be active in specific cell types of the transgenic animal. Thus, only in these cell types the env gene should be expressed and translated, and only in these cell types viral particles should be generated. However, after release of the viral particles comprising the amphotropic envelope protein the particles infect a lot—if not all—different cell types and different tissues, respectively, of the transgenic animal. Nevertheless, only newly infected cells in which the regulatory sequence is active release further viral particles, and so on. Those cells in which the regulatory sequence included in the vector is active can be detected easily, especially in case that the viral vector additionally comprises a marker gene as, e.g., a green fluorescent protein (gfp) gene. Accordingly, it can easily be evaluated if the regulatory sequence is indeed cell type specific or if the regulatory sequence is very unspecific. Thus, it can be decided if the regulatory sequence is indeed suitable for targeted gene therapy or not.

Another way to ensure that almost all cells of the transgenic animal are infectable with the viral vector comprising the regulatory sequence to be evaluated is to use modified surface proteins. These surface proteins could be modified to contain binding sites to ubiquitous cellular receptors.

In a preferred embodiment the transgenic animals are engineered to constitutively express in all tissues a specific receptor and the surface protein in the viral vector is engineered to present in the binding region the corresponding ligand. Alternatively, the animal could be engineered to constitutively express a ligand and the viral vector could be engineered to contain in the surface protein the corresponding receptor. In this alternative embodiment it might be necessary to provide the ligand with a cell membrane localization signal to ensure that the ligand remains bound to the cell membrane.

Thus, according to this embodiment the invention provides a method for the evaluation of the activity and/or specificity of regulatory elements comprising the steps of:

a) introducing into a cell of a transgenic non-human animal comprising in its genome one or more viral sequences, in particular viral genes, but being deficient in at least one viral gene required for generation of the virus, a viral vector comprising the at least one viral gene, wherein at least one of said viral genes in the viral vector is under control of the regulatory element to be evaluated, thereby allowing reconstitution of viral particle generation in the cells of the transgenic animal in which the regulatory element to be evaluated is active;

b) maintaining the transgenic animal under suitable conditions allowing the production of viral particles in cells of the transgenic animal; and,

c) detecting the cells, in which viral particles are produced.

The method according to the present invention allows to determine the “specificity of a regulatory sequence”. The term “specificity” in this context refers to the fact that regulatory elements might be active in all tissues in all or in specific stages of the animals development, active in specific tissues in all stages of the development or during certain stages of the development, only, or not active at all. Thus, “evaluation of the specificity of a regulatory element” means evaluation of the type and developmental stage of the cells in which the regulatory element is active. Only if the regulatory element is active in a specific cell, said cell will produce viral particles that are in turn able to infect other cells. Thus, the number of infected cells is increased (“snowball effect”) and under optimal conditions a large number of cells (and in an ideal case even all cells) of the animal should be infected. Methods are known to the person skilled in the art in order to determine in which cells the regulatory sequences are active. A convenient way to identify these cells is to include into the viral vector comprising the at least one viral gene under control of the regulatory element a marker gene that is under the control of the same regulatory element. This can be achieved by including into the viral vector separate expression cassettes coding for the at least one viral gene and the marker gene, respectively, wherein in both cassettes the gene expression is controlled by a copy of the same regulatory element. The disadvantage of this type of vector construct is that the regulatory sequence occurs twice in the vector genome which may lead to undesired recombination events. Thus, in the most preferred embodiment only one regulatory sequence controls the expression of both, the at least one viral sequence and the marker gene. In order to achieve the expression of both genes in one expression cassette it might be necessary to include an internal ribosome binding site (IRES) between the at least one viral gene and the marker gene. IRES elements are known to the person skilled in the art. In this context reference is made to the IRES elements of picornaviruses such as EMCV. Further IRES elements are disclosed below. Typical marker genes are the gene encoding the green fluorescent protein, the gene encoding β-galactosidase or genes encoding a protein to which specific antibodies bind. Methods to determine the expression of the marker genes are known to the person skilled in the art. The “activity” of a regulatory element can be evaluated by the quantification of the expression of the marker gene.

Moreover, the present invention provides a method for the rapid and easy analysis of viral components, especially of those components which should be used in viral vectors which, in turn, should be further used as safe vehicles in targeted gene therapy.

The cell tropism, i.e. the specificity of the viral infection, of enveloped viruses is determined by the viral envelope proteins that interact with corresponding receptors on the surface of cells. If the corresponding receptors are ubiquitous the virus is able to infect all cells. If the receptor is present only on specific cells, only these cells will be susceptible to infection. In the case of non-enveloped viruses the cell tropism is defined by specific proteins on the surface of the viral capsid.

In the context of the present invention the term “surface protein” refers to all viral proteins on the surface of a virus that interact with the target cells and that are necessary for viral entry into the cell to occur. Therefore this term covers the capsid proteins of non-enveloped viruses that come into contact with the cells as well as the envelope proteins of enveloped viruses. The term surface protein covers naturally occuring surface proteins as well as modified surface proteins. “Modified surface proteins” are surface proteins that have been modified to include binding sequences to cellular receptors that do not naturally occur in the viral surface protein. Examples for binding sequences not naturally occuring in the viral surface protein are receptor ligands (Kasahara et al. (1994), Science 266, 1373-1376), functional fragments thereof (Valsesia-Wittmann et al. (1996), J. Virol. 70, 2059-2064), the binding regions of monolclonal antibodies (Russell et al. (1993), Nucl. Acids Res. 21, 1081-1085) or binding regions of other viral surface proteins. The cited references disclose how such binding regions can be introduced into a viral surface protein to arrive in an infection of the target cells. The term “modified surface proteins” also covers surface proteins that have been modified to include binding sequences to cellular ligands lo that do not naturally occur in the viral surface protein. The term “cellular ligands” refers to ligands that are bound to the cell membrane. In this context this term covers naturally occuring cellular ligands.

The method according to the present invention can also be used for evaluating the specificitiy of a surface protein, in particular of an envelope protein of a virus, i.e. it can be analysed if a surface protein, in particular an envelope protein of a virus to be used as safe gene transfer vehicle, is indeed able to dock on the cell surface of special target cells, as, e.g., tumour cells only. In the following this is exemplified for the surface gene of an enveloped virus, i.e. for an env gene. A person skilled in the art will realise that the inventive concept can also be applied in a similar way to the genes of a non-enveloped virus that code for proteins required for the interaction with the cell. Consequently it is possible to substitute the terms “env-gene” and “Env-protein” by the more general term “surface gene” and “surface protein”, respectively. As pointed out above the term “surface protein” also covers “modified surface proteins” as defined above. If the gene the specificity of which is to be evaluated is an env gene, according to one embodiment of the invention the cells of the transgenic animal are deficient in the viral sequence encoding env, i.e. in cells of the transgenic animal either a non-functional Env protein or, in case that the env gene is completely deleted, no Env protein is produced. Correspondingly, the special env gene to be analysed is included in the viral vector, but preferably regulated by an ubiquitous, constitutively active regulatory sequence. The same also applies if the method according to the present invention is used to evaluate the distribution of a receptor for a ligand in an animal (see below). After infection or transduction of cells of the transgenic animal further viral particles comprising the envelope protein are generated and released. However, further cells of the transgenic animal are infected only if interaction occurs between the viral surface or envelope protein and the host cell membrane proteins which act as receptors. Subsequently, it can be evaluated if the envelope of the virus indeed comprises a limited infection spectrum and if it is, thus, applicable for targeted transfer of therapeutic genes into requisite target cells.

Thus, according to this embodiment the invention provides a method for the evaluation of the specificity of the surface protein of a virus comprising the steps of:

a) introducing into a cell of a transgenic non-human animal comprising in its genome one or more viral sequences, but being deficient in at least the viral sequence that comprises the gene which codes for the viral surface protein which is required for the generation of the virus, a viral vector comprising the at least viral sequence, thereby allowing reconstitution of the viral particle generation in the cell of the transgenic animal

b) maintaining the transgenic animal under suitably conditions allowing the production of viral particles in cells of the transgenic animal; and,

c) detecting the cells, in which viral particles are produced.

In an alternative of this embodiment the invention provides a method for the evaluation of the specificity of the surface protein of a virus comprising the steps of:

a) introducing into a cell of a transgenic non-human animal comprising in its genome at least the viral sequence comprising the gene which codes for the viral surface protein, but being deficient in at least one viral sequence required for generation of the virus, a viral vector comprising the missing viral sequences required for the generation of the virus, thereby allowing reconstitution of viral particle generation in the cell of the transgenic animal;

b) maintaining the transgenic animal under suitable conditions allowing the production of viral particles in cells of the transgenic animal; and

c) detecting the cells, in which viral particles are produced.

It is known to the person skilled in the art that this alternative embodiment will not work for viruses, in which the surface proteins produced from the cells interact with receptors on the cell surface to block the infection of viruses using the same receptors. It has been described that the env-protein of retroviruses might have this effect. I.e. it is known that cells expressing the retroviral env-gene are hardly infectable with a retrovirus having the corresponding env-protein on the viral surface. Thus, this alternative embodiment will only work for viral systems in which the produced surface protein does not block an infection with a virus having the same protein on the viral surface.

The method according to the present invention allows to determine the “specificity of a viral surface protein”. The term “specificity” in this context refers to the fact that viral surface proteins might interact with cellular receptors that are present on all types of cells or only on specific types of cells, during some or all stages of the animals development. Thus, “evaluation of the specificity of a viral protein” means evaluation of the type and developmental stage of the cells that can be infected with a virus having on the surface the surface protein to be evaluated. Only in the infected cells the production of viral particles occurs. Thus the production of viral particles indicates that the cell was infected with a virus having a protein on the viral surface that is specific for this cell. An alternative way to determine which cells have been infected is to include in the viral vector comprising at least the viral sequence which codes for the viral surface protein a marker gene expressing a detectable gene product. Examples for such marker genes are known to the person skilled in the art and include inter alia the gene encoding the green fluorescent protein or the β-galactosidase. The person skilled in the art is aware of methods allowing the detection and quantification of the expression of these genes. The marker genes should be controlled by a promoter that is preferably active in all cells and cell types. The viral vector may contain the marker gene and the viral sequence which codes for the viral surface protein in two separate expression cassettes that are controlled by the same or by different types of promoter. Preferably the gene encoding the viral surface protein and the marker gene are expressed in one expression cassette from one promoter. In this case an IRES element has to be inserted between both genes. The “activity” of a surface protein can be evaluated by the quantification of the amount of viral particles produced by a specific cell type or tissue or by the quantification of the expression of the marker gene.

As pointed out above the surface protein can also be a modified surface protein as defined above. In a preferred embodiment the modified surface protein is a surface protein that contains in the binding site, i.e. in the region that is accessible for binding to the cell, a ligand for a cellular receptor. Thus, the method according to the present invention can be used to evaluate in which cells the receptor for the ligand included in the binding site of the surface protein is expressed. Only those cells can be infected that contain a receptor specific for the ligand included in the viral surface protein. According to a preferred embodiment the unmodified surface protein is a surface protein that does not have a corresponding receptor on the surface of the cells of the animal. The virus with the unmodified surface protein should therefore not infect the cells of the animal. An example for this type of virus are ecotropic retroviruses. Alternatively, it is possible to insert the ligand sequence into a surface protein for which cellular receptors exist. In this case it is preferable to insert the ligand into the binding site of the surface protein in such a way that the surface protein as such cannot interact anymore with its cellular receptor. A binding of the modified viral surface protein to the cell then has to be due to the interaction of the ligand in the surface protein with the cellular receptor of the ligand. The method according to the present invention can also be applied if the surface protein portion of the modified surface protein shows a residual binding to the cellular receptor of the unmodified surface protein. In this embodiment it will be necessary to infect the transgenic animal with a vector expressing the unmodified surface protein and to compare the results with the results obtained after infection of the same type of transgenic animal with the vector expressing the modified surface protein.

In the context of the present invention the term “ligand” refers to any peptide or protein which specifically binds to a specific structure on the cellular surface, i.e. a receptor. Thus, a ligand can be e.g. a growth factor, a hormone, such as insulin or a binding site of a monoclonal antibody. A “receptor” is defined as a structure on the cellular surface that interacts with a ligand. Thus, a receptor can be inter alia a growth factor receptor, a hormone receptor or a antigen specific to the antigen binding site in the modified viral surface protein. The term “distribution of a receptor in an animal” refers to the type of cells and tissues on which the receptor is expressed and present on the surface of a cell. Thus, the evaluation of the distribution of a receptor in an animal is in fact the evaluation of the type of cells and tissues having the receptor on the cell surface.

In this embodiment the invention provides a method for the evaluation of the distribution of a receptor for a ligand in an animal comprising the steps of:

a) introducing into a cell of a transgenic non-human animal comprising in its genome one or more viral sequences, but being deficient in at least the viral sequence that comprises the gene which codes for the viral surface protein which is required for the generation of the virus, a viral vector comprising the at least viral sequence, thereby allowing reconstitution of the viral particle generation in the cell of the transgenic animal, wherein the viral sequence coding for the viral surface protein contains the coding sequence for the ligand so that upon expression of this sequence a modified viral surface protein is produced that contains the sequence of the ligand in the part of the protein that is accessible for interaction with a cellular receptor

b) maintaining the transgenic animal under suitably conditions allowing the production of viral particles in cells of the transgenic animal; and,

c) detecting the cells, in which viral particles are produced

In an alternative of this embodiment the invention provides a method for the evaluation of the distribution of a receptor for a ligand in an animal comprising the steps of:

a) introducing into a cell of a transgenic non-human animal comprising in its genome at least the viral sequence that comprises the gene which codes for the viral surface protein, but being deficient in at least one viral sequence required for the generation of the virus, a viral vector comprising the missing viral sequences required for the generation of the virus, thereby allowing reconstitution of the viral particle generation S in the cell of the transgenic animal, wherein the viral sequence coding for the viral surface protein contains the coding sequence for the ligand so that upon expression of this sequence a modified viral surface protein is produced that contains the sequence of the ligand in the part of the protein that is accessible for interaction with a cellular receptor;

b) maintaining the transgenic animal under suitably conditions allowing the production of viral particles in cells of the transgenic animal; and,

c) detecting the cells, in which viral particles are produced.

According to this alternative embodiment the expression of the modified viral surface protein should preferably be regulated by an ubiquitous constitutively active regulatory sequence. Preferred regulatory sequences have been mentioned. Most preferred are the MLV regulatory sequences.

As already pointed out above it is known to the person skilled in the art that this alternative embodiment will not work for viruses if the modified surface proteins produced from the cells interact with receptors on the cell surface to block the infection of viruses using the same receptors. Thus, this alternative embodiment will only work for viral systems in which the modified surface protein produced from the uninfected cells does not block an infection with a virus having the same protein on the viral surface..

In general the most prefered embodiments of the method for the evaluation of the distribution of a receptor for an animal as summarized in the two alternatives above correspond to the most preferred embodiments of the method for the evaluation of the specificity of a viral component, wherein the viral component is a viral surface protein.

According to a further embodiment of the invention, the transgenic animal is deficient in the viral sequence coding for the packaging signal; most preferably, this viral sequence is not functional at all within the genome of the transgenic animal.

As already briefly indicated above, the most preferred transgenic non-human animal used in the method according to the present invention is a mouse as, e.g., of the strain CD1, FVB, NMRI, C57/Black6, ICR. The animal may be healthy or suffer from a genetic, metabolic, proliferative or any other relevant disorder. Most preferably, the transgenic mouse lacks an efficient immune system as it is the case, e.g., in a severe combined immune deficient (SCID) mouse. Accordingly, the immune system of the animal can not attack infectious particles produced.

The viral sequence(s) integrated into the genome of the transgenic animal and the viral vector comprising the viral sequence(s) for reconstitution of viral particle generation are preferably derived from the same viral system, i.e., the viral sequences as well as the viral vector are either derived from non-retroviruses or retroviruses. Most preferably, the viral sequences and the viral vector are of retroviral origin.

Retroviral vectors are based on a retroviral genome. The retroviral genome of simple retroviruses consists of an RNA molecule basically comprising the structure R-U5-gag-pol-env-U3-R. During the process of reverse transcription, the U5 region is duplicated at the right hand end of the generated DNA molecule, whilst the U3 region is duplicated and placed at the left hand end of the generated DNA molecule. The resulting structure U3-R-U5 is called LTR (Long Terminal Repeat) and is thus identical and repeated at both ends of the DNA structure or provirus. The U3 region at the left-hand end of the provirus harbours the promoter. This promoter drives the synthesis of an RNA transcript initiating at the boundary between the left-hand U3 and R regions and terminating at the boundary between the right hand R and U5 region. This RNA is packaged into retroviral particles and transported into the target cell to be infected. In the target cell the RNA genome is again reverse transcribed as described above.

The typical retroviral vector comprises two complete LTRs—a 5′ and a 3′ LTR—and an insertion site for the sequence of interest in between the LTRs in the body of the vector. In case, that a regulatory sequence shall be analysed, both, the regulatory sequence as well as the retroviral sequence and gene, respectively, regulated by said regulatory sequence can be inserted into the body of the vector. However, preferably, the regulatory sequence is inserted into an LTR, more preferably into the U3-region of an LTR. Most preferably, the retroviral vector is a promoter conversion (ProCon) vector (see PCT/EP95/03445).

In the promoter conversion vector the right hand U3 region is altered, but the normal left hand U3 structure is maintained; the vector can be normally transcribed into RNA utilizing the normal retroviral promoter located within the left hand U3 region. However, the generated RNA will only contain the altered right hand U3 structure. In the infected target cell, after reverse transcription, this altered U3 structure will be placed at both ends of the retroviral structure.

The altered region carries a polylinker instead of the U3 region. Thus, any regulatory sequence, including those directing tissue specific expression can be easily inserted. This promoter is then utilized exclusively in the target cell for expression of linked genes carried by the retroviral vector.

According to this embodiment of the invention, the retroviral vector comprising the retroviral gene under the control of the regulatory sequence of interest can be produced as follows: In a ProCon vector a gene encoding a retroviral protein, preferably env, is present in the body of the vector. The regulatory sequence of interest is inserted into the U3 region of the 3′-LTR and the resulting vector is transfected into a packaging cell. According to the ProCon principle, in the packaging cell line the expression of the retroviral vector is regulated by the normal unselective retroviral promoter contained in the U3 region of the 5′-LTR. Subsequently, the vector is packaged into a retroviral particle. However, when a cell of the transgenic animal, in this example expressing gag and pol, is infected with said retroviral particles, promoter conversion occurs, and the expression of env is regulated by the regulatory sequence of interest. According to the present invention, production of retroviral particles will occur only in cells in which the regulatory sequence is active.

Preferably, in the ProCon vector the regulatory sequences are inserted within the U3 region of the 3′ LTR. More preferably, the U3 region is wholly or partly replaced by the regulatory sequence. Nevertheless, the regulatory sequence can also be inserted between U3 and the R region of the 3′ LTR.

The LTRs of the retroviral vector used for the infection or transduction of the transgenic animal according to the present invention are preferably selected from an element of the group comprising the LTR of Murine Leukemia Virus (MLV), Mouse Mammary Tumor Virus (MMTV), Murine Sarcoma Virus (MSV), Simian Immunodeficiency Virus (SIV), Human Immunodeficiency Virus (HIV), Human T-cell Leukemia Virus (HTLV), Feline Immunodeficiency Virus (FIV), Feline Leukemia Virus (FELV), Bovine Leukemia Virus (BLV), Mason-Pfizer-Monkey virus (MPMV) and Rous Sarcoma Virus (RSV). Most preferably, both LTRs are derived from the Murine Leukemia Virus (MLV).

The env gene regulated by the regulatory sequence to be evaluated may be VSV-g, LCMV-gp (glycoprotein of the lymphocytic choriomeningitis virus) or RD114, which allows infection of cells of different species, i.e., which gives a wide host range. More preferably env is derived from a Murine amphotropic env, i.e. an env infecting both, mouse and non-murine species. Most preferably, the amphotropic 4070A env of MLV is used.

According to another preferred embodiment of the invention, the retroviral gene(s) in the transgenic animal and the retroviral gene(s) in the retroviral vector are derived from the same type of retrovirus. More preferably, the LTRs of the retroviral vector are also derived from the same origin as the genes. Most preferably the retroviral genes and the LTRs are derived from MLV.

According to another embodiment of the invention, the retroviral vector comprises in addition to the retroviral gene a heterologous coding sequence. The term “heterologous” is used for any combination of DNA sequences that is not normally found intimately associated in nature. The heterologous coding sequences are preferably selected from an element of the group comprising marker genes, therapeutic genes and/or anti-tumor genes. Said marker and therapeutic genes are further preferably selected from an element of the group comprising β-galactosidase gene, neomycin gene, luciferase gene, Herpes Simplex Virus thymidine kinase gene, puromycin gene, cytosine deaminase gene, hygromycin gene, cytochrome P450 gene, fluorescence marker genes like green fluorescent protein (gfp) gene, blue fluorescent protein (bfp) or yellow fluorescent protein (yfp) gene, and zeocine resistance gene.

Most preferably, the additional heterologous gene is operatively linked to an internal ribosome entry site (IRES). An IRES promotes the attachment of ribosomes to a motive within an mRNA sequence, thereby allowing the translation of the corresponding gene. According to this embodiment of the invention the retroviral vector comprises a retroviral gene and an additional heterologous gene linked to the IRES. Consequently, though transcription of the retroviral gene(s) and also of the additional heterologous gene(s) are both regulated by the same regulatory sequence, further translation of the expressed mRNA into protein occurs independently and separately, respectively.

In principle, all regulatory sequences can be analyzed by the method according to the present invention. Regulatory sequences may comprise but are not limited to an element of the group comprising Whey Acidic Protein (WAP), Mouse Mammary Tumor Virus (MMTV), β-lactoglobulin, lactalbumine and casein specific regulatory elements and promoters which may be used to target human mammary tumors. In addition, pancreas specific regulatory elements and promoters, such as carbonic anhydrase promoter, glucokinase promoter and phosphoglycerate kinase promoter, lymphocyte specific regulatory elements and promoters including human immunodeficiency virus (HIV), immunoglobulin and MMTV lymphocytic specific regulatory elements and promoters and MMTV specific regulatory elements and promoters such as ^{MMTV}P2 conferring responsiveness to glucocorticoid hormones or directing expression to the mammary gland, T-cell specific regulatory elements and promoters such as T-cell receptor gene and CD4 receptor promoter, B-cell specific regulatory elements and promoters such as immunoglobulin promoter or mb1 and tumor specific promoters such as the tissue factor promoter, the carcino embryonic antigen (CEA) promoter and the vascular endothelial growth factor (VEGF) promoter.

The method according to the present invention is preferably used to analyze a heterologous regulatory sequence in the context of a retrovirus intended to be used for gene therapy. In this case, the regulatory sequence drives the retroviral gene instead of or in addition to the heterologous sequence used for gene therapy. Advantageously, the regulatory sequence of interest driving the expression of the retroviral gene functions exactly the same way as in the retroviral vector for gene therapy when the regulatory sequence drives the expression of a marker or therapeutic gene. Hence, the method according to the present invention allows to predict the function of the regulatory sequence during gene therapy, e.g. to predict the level of activity and the tissue specificity of the regulatory sequence.

The present invention also relates to a transgenic non-human animal obtainable by the method according to the invention and provides a transgenic non-human animal applicable in the method according to the present invention. The latter one comprises in its genome one or more viral sequences but is deficient in at least one viral sequence required for generation of the virus. Accordingly, the cells of the transgenic animal produce viral proteins but the retroviral gene(s) can not be incorporated into retroviral particles since a sequence required for replication and/or packaging—such as the psi packaging signal and/or an LTR-region—is mutated or deleted. Introduction of the sequence required for generation of viral particles should not only allow reconstitution of viral particle generation, but also dissemination of said viral particles in the transgenic animal.

Preferably, the transgene(s) Pre expressed in a lot of different cell types, most preferably in all cell types. Hence, according to a preferred embodiment of the invention, the expression of the viral transgene is driven by an ubiquitous, constitutively active regulatory sequence. In case that the viral sequences included in the transgenic animal genome are of retroviral origin, expression of said retroviral genes are preferably regulated by the SV40 enhancer and/or promoter, the β-actin promoter, the ROSA26 promoter, the CDC10 promoter or the ubiquitin promoter, and most preferably by the Murine Leukemia Virus (MLV) promoter.

Preferably, the animal is transgenic for gag and/or pol. More preferably, gag and/or pol is derived from MLV and most preferably the expression of gag and/or pol derived from MLV is regulated by an MLV promoter.

### SUMMARY OF THE INVENTION

The invention inter alia comprises the following, alone or in combination:

A method for the evaluation of the activity and/or specificity of a regulatory sequence or of viral component comprising the steps of:

a) introducing into a cell of a transgenic non-human animal comprising in its genome one or more viral sequences, but being deficient in at least one viral sequence required for generation of the virus, a viral vector comprising the at least one viral sequence, thereby allowing reconstitution of viral particle generation in tile cell of the transgenic animal;

b) maintaining the transgenic animal under suitable conditions allowing the production of viral particles in cells of the transgenic animal; and,

c) detecting the cells, in which viral particles are produced.

A method for the evaluation of the distribution of a receptor for a ligand in an animal comprising the steps of:

a) introducing into a cell of a transgenic non-human animal comprising in its genome one or more viral sequences, but being deficient in at least the viral sequence that comprises the gene which codes for the via surface protein which is required for the generation of the virus, a viral vector comprising the at least viral sequence, thereby allowing reconstitution of the viral particle generation in the cell of the transgenic animal, wherein the viral sequence coding for the viral surface protein contains the coding sequence for the ligand so that upon expression of this sequence a modified viral surface protein is produced that contains the sequence of the ligand in the part of the protein that is accessible for interaction with a cellular receptor;

b) maintaining the transgenic animal under suitably conditions allowing the production of viral particles in cells of the transgenic animal; and,

c) detecting the cells, in which viral particles are produced.

The method as above, wherein the viral component is a viral surface protein;

the method as above, wherein the transgenic non-human animal is a mammal, preferably a rodent, most preferably a mouse;

the method as above, wherein the transgenic non-human animal is a severe combined immune deficient (SCID) mouse;

the method as above, wherein the transgenic animal is deficient in the viral sequence coding for the packaging signal;

the method as above, wherein the viral sequence coding for the packaging signal is deleted;

the method as above, wherein the expression and/or translation of the viral sequence(s) included in the genome of the transgenic animal are regulated by an ubiquitous, constitutively active regulatory sequence;

the method as above for the evaluation of the activity and/or specificity of a regulatory sequence, wherein expression and/or translation of the at least one viral sequence included in the vector is regulated by the regulatory sequence to be analysed;

the method as above, wherein expression and/or translation of the gene encoding env is regulated by the regulatory sequence to be analysed;

the method as above, wherein the gene encoding env codes for an amphotropic env;

the method as above, wherein the regulatory sequence to be analysed is suitable for targeted gene therapy;

the method as above for the evaluation of the specificity of the envelope protein of a virus, wherein the transgenic animal is deficient in the viral sequence encoding env;

the method as above, wherein the viral sequence encoding env is deleted;

the method as above, wherein expression and/or translation of the env gene included in the viral vector is regulated by an ubiquitous, constitutively active regulatory sequence;

the method as above, wherein the envelope protein is suitable for targeted gene therapy;

the method as above, wherein the viral sequences and the viral vector are of retroviral origin;

the method as above, wherein the transgenic animal is transduced with the retroviral vector and/or infected with a retroviral particle comprising said retroviral vector;

the method as above, wherein the retroviral sequences of the retroviral vector and the retroviral sequences included in the genome of the transgenic animal are derived from the same type of retrovirus;

the method as above, wherein the retroviral sequences are derived from Murine Leukemia Virus (MLV);

the method as above, wherein for the evaluation of the activity and/or specificity of a regulatory sequence the regulatory sequence is inserted into a Long Terminal Repeat (LTR) of the retroviral vector.

the method as above, wherein the regulatory sequence is inserted into a U3-region of the LTR.

the method as above, wherein the retroviral vector is based on a promoter conversion vector;

the method as above, wherein the retroviral vector comprises in addition to the retroviral sequence(s) a heterologous gene;

the method as above, wherein the heterologous gene is a therapeutic gene, an anti-tumour gene and/or a marker gene;

the method as above, wherein the marker gene is a green fluorescent protein (gfp) gene and/or a zeocine resistance gene;

the method as above, wherein the heterologous gene is operatively linked to an internal ribosomal entry site;

a transgenic non-human animal obtainable by the method as above;

a transgenic non-human animal comprising in its genome one or more viral sequences, but being deficient in at least one viral sequence required for generation of the virus, wherein an introduction of the at least one viral sequence into a cell of the transgenic animal allows reconstitution of viral particle generation and dissemination of said viral particles in the transgenic animal;

the transgenic animal as above, wherein the transgenic non-human animal is a mammal, preferably a rodent, most preferably a mouse;

the transgenic animal as above, wherein the mouse is a severe combined immune deficient (SCID) mouse;

the transgenic animal as above, being deficient in the viral sequence encoding env.

the transgenic animal as above, wherein expression and/or translation of the viral sequences included in the genome of the transgenic animal are regulated by an ubiquitous, constitutively active regulatory sequence;

the transgenic animal as above, wherein the viral sequences are of retroviral origin;

the transgenic animal as above, wherein the regulatory sequence is selected from an element of the group comprising SV40 enhancer and/or promoter, β-actin promoter, ROSA26 promoter, CDC10 promoter, ubiquitin promoter and/or MLV promoter;

the transgenic animal as above, wherein the retroviral sequences are derived from Murine Leukemia Virus (MLV);

use of the transgenic animal as above in the method as above.

### FIGURE

FIG. 1 is a schematic representation of intermediate cloning plasmids and semi-replicative retroviral vectors. Abbreviations: CMV: immediate early promotor of the human cytomegalovirus; env: viral envelope gene; eGFP: gene for the enhanced green fluorescent protein; IRES: internal ribosome entry site of the Encephalo myocarditis virus; SV40-neo (neomycin resistance gene under the control of the SV40 promoter/enhancer); gfp/zeo: fusion of the gene coding for the green fluorescent protein and the zeocin resistance gene; cyp/zeo: fusion of the (cytochrom p450 2B1-zeocin resistance fusion gene); LTR: long terminal repetition; Ψ, retroviral packaging signal MMTV: mouse mamma tumour virus

### EXAMPLE(S)

The following example(s) will further illustrate the present invention. It will be well understood by a person skilled in the art that the provided example(s) in no way may be interpreted in a way that limits the applicability of the technology provided by the present invention to this example(s).

1. Construction of MLV Based Semi-Replicating Retroviral Vectors pLEIGZ, pLEIGZM, pLEICZ and pLEICZM

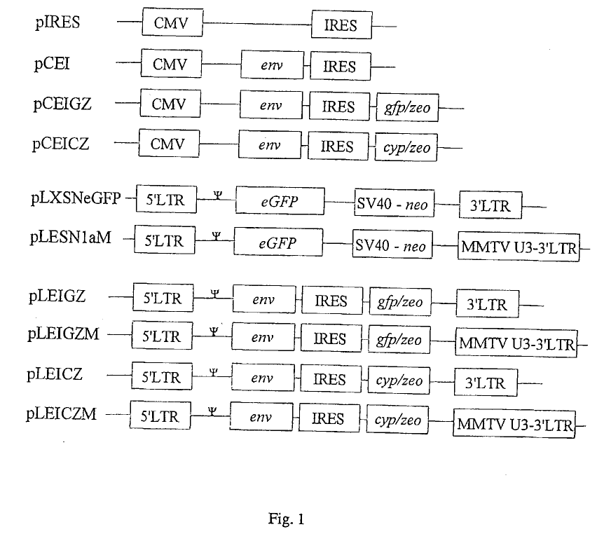

The semi-replicative retroviral vectors (FIG. 1) were derived from a number of different cloning and intermediate vectors (FIG. 1).

To obtain the first intermediate construct, the basic cloning vector pIRES (Invitrogen), carrying the internal ribosome entry site from EMCV (Encephalo myocarditis virus), was linearized with MIuI and ligated to the 2118 bp env-gene-fragment resulting from an AccI/PstI digestion of plasmid pALF (Cosset, F. -L., Takeuchi, Y., Battini, J. -L., Weiss, R. A., Collins, M. K. L. 1995; Journal of Virology 69, 7430-7436). This first intermediate construct was termed pCEI with 8214 bp of length.

Next, to create vectors pCEIGZ and pCEICZ, the precursor plasmid pCEI was linearized with XbaI to insert a gfp/zeo-cassette or a cyp/zeo-cassette, respectively. The gfp/zeo fusion gene cassette was obtained by digesting the expression vector pTracerSV40 (Invitrogen) with enzymes RsrII/BseRI and isolating a 1148 bp fragment. In parallel, the cyp/zeo-fusion gene was created containing the rat cytochrome P450 2B1 gene from nucleotide 25 to 1458 (Genbank accession number M37134) fused to Streptoallteichus hindustanus bleomycin gene from nucleotides 15 to 377 (Genbank accession number X90639). Insertion of the gfp/zeo-cassette into the linearized plasmid pCEI resulted in vector pCEIGZ, insertion of the cyp/zeo-cassette into the linearized plasmid pCEI in vector pCEICZ.

To create plasmid pLEIGZ, pCEIGZ was digested with PstI/NotI resulting among others in a 4185 bp fragment containing the env-IRES-gfp/zeo cassette. In parallel, the retroviral vector pLXSNeGFP (Klein, D., Indraccolo, S., von Rombs, K., Amadori, A., Salmons, B., Günzburg W. H. 1997; Gene Therapy 4, 1256-1260) was digested with RstII/EcoRI, the obtained 4808 bp fragment was blunted with T4 DNA Polymerase (Life Technologies), following the protocol of the manufacturer, and ligated to the 4185 bp fragment.

In parallel to the construction of plasmid pLEIGZ a ProCon based version (Saller, R. M., Öztürk, F., Salmons, B. Günzburg, W. H. 1998; Journal of Virology 72, 1699-1703) was constructed. This plasmid, called pLEIGZM, is very similar to pLEIGZ, but carries the heterologous MMTV promoter region instead of the MLV-U3 region in the 3′-LTR. Thus, expression of genes within the retroviral vector is driven by the MLV-promoter in pLEIGZM transfected cells, but by the MMTV promoter in LEIGZM infected cells. Plasmid pLEIGZM has been constructed using the 4185 bp PstI/NotI fragment of pCEIGZ in a ligation reaction with the 5283 bp fragment derived from an RsrII/EcoRI restriction digest of plasmid pLESN1aM. This plasmid was obtained by PCR-amplification of an 1025 bp neomycin-gene containing fragment of pLXSNeGFP using primers Bi01 (5′-GCCTCGGCCTCTGAGCTATT-3′) and Bi02 (5′-ATATCCGCGGGCTAGCTTGCCAAACCT-3′), cutting of this fragment with SacII and HindIII and ligating it into the 5787 bp SacII/HindII-fragment of pLXPCMTVeGFP. This plasmid was generated by inserting the 862 bp SmaI/HpaI-fragment of pEGFP-1 (Clontech) containing the eGFP-gene into plasmid pLXPCMTV linearized with HpaI. Plasmid pLXPCMTV was obtained by partially digesting plasmid pLXI25 with SacII, treatment of the linearized vector with T4 polymerase and religation. Thereby, the SacII site in the 5′-end of the 5′-LTR was destroyed and the remaining SacII site in the 5′-end of the 3′-LTR became unique. Plasmid pLX125 was created by ligating the 3545 bp BamHI/AfIIII-fragment of plasmid pLXSN (Miller, A. D., Rosman, G. J. 1989, Biotechniques 7: 980-990) with the 4263 bp BamHI/Af/II-fragment of plasmid p125.6 (Saller, R. M. 1994, Doctoral Thesis, Ludwig-Maximilian University, Munich, Germany).

To create the cytochrome P450 2B1-gene containing vectors pLEICZ and pLEICZM, plasmid pCEICZ was digested with the restriction enzymes NheI and NotI resulting in a 4663 bp fragment. This fragment, containing the env-IRES-cyp/zeo cassette, was ligated to a 4808 bp RsrII/EcoRI-fragment of plasmid pLXSNeGFP resulting in vector pLEICZ. Ligation of the env-IRES-cyp/zeo cassette to a 5283 bp fragment derived from a RsrII/EcoRI restriction digest of plasmid pLESN1aM results in vector pLEICZM.

All restriction digests, blunt ending and ligation reactions were performed using enzymes from Life Technologies following the protocol of the manufacturer. Transformations were performed, according to the instructions of the manufacturer, with E. coli ME DH5α bacteria obtained also from Life Technologies.

All constructs were analysed in vitro for expression of the env-gene by mobilisation assays: 5×10⁵ 293 gagpol semi-packaging cells, which were obtained by transfection of 293 cells (ATCC CRL 1573) with pGagPoIGPT (Markowitz, D., Goff, S., Bank, A 1988; Journal of Virology 62, 1120-1124), were transfected with 3 μg of the vector to be tested and with 3 μg of pLXSNeGFP. Transfection was performed using the calcium-phosphate transfection method according to the instructions of the supplier (Amersham Pharmacia). In case the env-gene is expressed, viral particles carrying the vector PLXSNeGFP as their genome should be obtained. 24 h after transfection cells were washed twice with phosphate buffered saline and 3 ml of fresh medium (DMEM+5% FCS) was added. 48 h after transfection, 1 ml of medium supernatant was used to infect 4×10⁵ NIH/3T3 target cells (ATCC CRL-1658). Number of infected cells were analysed after 48 h via FACS (Table 1).

| Producer | Transfection | FACS1 | infection | FACS2 |
|---|---|---|---|---|
| 293gp9T | untransfected | 0% | NIH3T3 | 0% |
| 293gp9T | mock | 0% | NIH3T3 | 0% |
| 293gp9T | pLXSNeGFP | 55% | NIH3T3 | 0% |
| 293gp9T | pALF + pLXSNeGFP | 28% | NIH3T3 | 1% |
| 293gp9T | pCEI + pLXSNeGFP | 13,6% | NIH3T3 | 1,8% |
| 293gp9T | pCEICZ + pLXSNeGFP | 24,8% | NIH3T3 | 1,9% |
| 293gp9T | pCEIGZ + pLXSNeGFP | 32% | NIH3T3 | 2,4% |
| 293gp9T | pLEIGZ + pLXSNeGFP | 45,8% | NIH3T3 | 2,5% |
| 293gp9T | pLEIGZ | 0,02% | NIH3T3 | 0% |

(Table 1, explanation: 293 gp9T semipackaging cells were transfected with plasmids as indicated. At time of infection of NIH cells using the virus containing supernatant of the transfected cells, these cells were subjected to FACS analysis (FACS 1). FACS analysis of infected cells (FACS 2) was performed 48 h later. The numbers indicate the percentage of green (gfp-positive cells)).

Function of the IRES element and the gfp/zeo-fusion cassette was evaluated via selection of zeocin-resistant colonies. A population of at least 100 clones was established and subjected to FACS analysis. Interestingly, 63% of the zeo-resistant cells express the gfp-gene.

2. Production of Retroviral Particles

Production of recombinant retroviral particles was performed by transfection of 5 μg plasmid DNA into the 293 gag/pol semipackaging cells, which were obtained by transfection of 293 cells (ATCC CRL 1573) with pGagPoIGPT, or with conventional packaging cells like PA317 (Miller, A. D., Buttimore, C. 1986, Molecular and Cellular Biology 6: 2895-2902). For that purpose, 5×10⁵ packaging cells were seeded in 3 cm dishes 24 h prior to transfection. Transfection was performed according to the protocol provided by the manufacturer (CellPhect Transfection Kit; Amersham Pharmacia). 18 h later, medium was removed, cells were washed twice with PBS and fresh medium was added. 48 h after transfection the cells were trypsinized and diluted for selection to obtain clones and/or populations stable producing recombinant retroviral particles.

For infection experiments, 1×10⁶ producer cells were seeded into 3 cm dishes 48 h prior to infection. 24 h later medium was removed and one ml of fresh medium was added. In addition, at this time point 2×10⁵ recipient cells were plated in a 3 cm dish. Infection was performed using one ml of virus containing medium filtered through a 45 μm filter onto the target cells. Polybrene was added to a final concentration of 8 μg/ml medium. 6 h after infection 3 ml of fresh medium were added to each dish. Expression of the eGFP reporter gene was measured 72 h after infection by FACS analysis.

3. Construction of the gag/pol Expression Construct pTopoGagPoI

The expression vector pTOPOgagpoI was constructed by ligation of the MLV gag/pol expression cassette derived by long template PCR of plasmid pGagPoIGPT with the cloning vector pCR-XL-TOPO (Invitrogen). Expression of the gag/pol coding region of plasmid pGagPoIGPT (Markowitz, D., Goff, S., Bank, A. 1988, Journal of Virology 62: 1120-1124) is regulated from the MLV LTR-promoter region 5′-fused to a 412 bp sequence homologue to the rat lipocortin enhancer/promoter. This expression cassette was isolated by long template PCR using primers primer GPTransHin (5′-CTGTGATAAACTAGGGCATTA-3′) specific to the beginning of the lipocortin enhancer/promoter and GPTransRueck2 (5′-GTTTATTGCAGCTTATAATGG-3′) specific to the end of the polyadenylation signal. PCR resulted in a 6731 bp fragment. The fragment was purified by running on a 0.8% agarose gel, the DNA band was excised and DNA eluted using the Qiaquick protocol (Qiagen). 40 ng of the PCR-fragment and 10 ng of the cloning vector pCR-XL-TOPO were mixed and incubated for 5 minutes at room temperature. Ligation was stopped by adding one μl 6× TOPO cloning stop solution. “One shot” competent bacteria (Invitrogen) were transformed with the cloning mix and kanamycin resistant colonies selected. Plasmid DNA was prepared and test digested with the restrictions enzymes EcoRI, XhoI, XbaI, SpeI, respectively. The final correct plasmid was designated pTOPOGagPoI.

To evaluate the functionality of this plasmid, NIH and HeLa cells were transfected with 5 μg of plasmids pTOPOGagPoI, pALF (Cosset, F. -L., Takeuchi, Y., Battini, J. -L., Weiss, R. A., Collins M. K. L. 1995, Journal of Virology 69: 7430-7436) comprising an MLV env expression cassette and pLXSNeGFP (Klein, D., Indraccolo, S., von Rombs, K., Amadori, A, Salmons, B., Günzburg, W. H. 1997, Gene Therapy 4: 1256-1260) comprising an eGFP-expressing retroviral vector. 48 h later 4×10⁵ NIH and HeLa cells were infected with the media supernatant of the transfected cells. Viral titer was measured 72 h later by FACS analysing eGFP expression. Experiments using plasmid pTOPOgalpoI gave rise to same titer values as experiments using plasmid pGagPoIGPT as a control, indicating full function of the gag/pol expression cassette in vector pTOPOGagPoI.

4. Generation of gag/pol Transgenic Mice

To establish gag/pol transgenic mice plasmid pTOPOGagPoI was digested with NsiI to isolate said gag/pol expression cassette. The digestion mixture was purified on 0.8% agarose gel and the 6845 bp fragment was excised and the DNA eluted using the QiaExII gel extraction kit (Qiagen). Subsequently the DNA was precipitated with 12% PEG6000, 1.5 M NaCl, resuspended in ddH₂O and stored at −20 ° C.

Male B6CBAF-1 hybrid mice were vasectomized at an age of 8-10 weeks and two weeks later each male was crossbred with two 5-8 weeks old female CD-1 mice without hormone treatment. Phantom pregnant recipient mice were used for DNA-injected zygotes.

To provide superovulated donors 7.5 I.E. PMSG (pregnant mare serum gonadotropin, eCG) in 150 μl NaCl solution were injected intraperitoneally into 4-5 weeks old female CD-1 mice. 48 h later ovulation was triggered with 5 I.E. hCG and the donor females were crossbred with CD-1 mice. 16 h after hCG (human chorionic gonadotropin) treatment the crossbred females were euthanised. Fertilized zygotes were prepared via separation of kumulus cells using 1 mg/ml hyaluronidase in PBS/10% FCS for 3 min at 37° C. and subsequently of unfertilized eggs. Zygotes were-stored at 37° C. in a drop of PBS/2% FCS covered by paraffine oil.

One to 2 pl microinjection solution (1000-2000 DNA-copies) were injected into the nucleus of each zygote using a glass capillary. 12 to 15 microinjected zygotes were transferred into the ovary duct of each donor mouse. Transgenicity of the offspring mice was analysed as described in the following.

Transgenic lines were established after crossbreeding of positive founder mice with CD1 wildtype animals.

Screening of putative transgenic mice was performed via PCR amplification of the lipocortin-LTR-gag sequence from mouse genomic DNA. Mouse tail ends were clipped and genomic DNA prepared using the salting out procedure by Miller et al. (Miller, S. A., Dykes, D. D., Poleky, H. F. 1988, Nucleic Acids Research 16, 215). Finally DNA was dissolved in 300 μl of sterile water. One μl was subjected to PCR using primers gagpol-screen (5′-TGCGCTGCTGAGAAGCCAGT-3′) and gagpol-screen2 (5′-TTGTGAGCGATCCGCTCGAC-3′). Positive samples gave rise to a specific 1115 bp signal in agarose gelelectrophoresis.

In addition, selected samples were subjected to Southern blot-analysis. 20 μg of DNA were digested with SspI/NotI, separated by agarose gelelectrophoresis and transferred onto a nylon membrane (Zeta Probe, Biorad). Subsequently, the blotted gel was stained with ethidium bromide to estimate transfer efficiency. The membrane was washed three times for 10 minutes with 2×SSC (3 M NaCl, 0.3 M Tri-sodium-citrate x 2H₂O, pH 7.5-8.0) and air-dryed. The DNA was fixed onto the membrane by UV cross-linking. After prehybridisation (6×SSC; 5× Denhardt's; 0.5% SDS; 50 μg/ml salmon sperm DNA) at 68° C. for 2 hours the membrane was hybridised overnight at 68° C. using a transgene-specific probe. Subsequently, the membrane was washed twice with 2×SSC, 0.1% SDS for 20 minutes at 68° C. and once with 0.1×SSC, 0.1% SDS for 15 minutes at 68° C. At last the membrane was exposed to a phosphorimager plate (Fuji).

To obtain the transgene-specific probe, plasmid pTOPOgag/poI was digested with SspI and NotI generating among others a 2088 bp fragment carrying part of the lipocortin promoter fragment, the MLV promoter and part of the gag coding region. The fragment was purified by agarose gelelectrophoresis and 50 ng of the fragment were labeled by random priming with alpha-³²P-dCTP using Random Primers DNA Labeling System kit (Life technologies) following the protocol provided by the manufacturer. The labeled probe was purified via a BioSpin-30 column (BioRad) according to manufacturer's protocol. To obtain a sufficient signal after hybridisation a minimal specific activity of 4×10⁸ cpm/μg DNA was used for the hybridisation reaction.

We claim:

1. A method for the evaluation of at least one property selected from the group consisting of activity and specificity of a regulatory sequence comprising the steps of:
a) using a transgenic non-human animal comprising in its genome one or more viral sequence, but being deficient in one or more viral sequence required for generation of infectious virus particles;
b) introducing into said transgenic non-human animal the one or more viral sequence required for generation of infectious virus particles under control of the regulatory sequence to be evaluated, thereby allowing reconstitution of infectious viral particle generation within cells of said transgenic non-human animal in which the regulatory sequence is active;
c) maintaining the transgenic animal under suitable conditions allowing the production and release of infectious viral particles in cells of said transgenic non-human animal in which the regulatory sequence is active; and
d) detecting at least one site selected from the group consisting of cells and tissues in which viral particles are produced,
wherein the production of viral particles by a cell or tissue indicates that the regulatory sequence is active in the cell or tissue.

2. A method for the evaluation of at least one property selected from the group consisting of activity and specificity of a surface protein comprising the steps of:
a) using a transgenic non-human animal comprising in its genome one or more viral sequence, but being deficient in one or more surface protein sequence required for generation of infectious virus;
b) introducing into said transgenic non-human animal the one or more surface protein sequence under control of a ubiquitous regulatory element, thereby allowing reconstitution of viral particle generation within cells of the transgenic non-human animal for which the surface protein is functional;
c) maintaining the transgenic animal under suitable conditions allowing the production and release of infectious viral particles in cells of said transgenic non-human animal for which the surface protein is functional; and
d) detecting at least one site selected from the group consisting of cells and tissues in which viral particles are produced,
wherein the production of viral particles by a cell or tissue indicates that the surface protein is functional in the cell or tissue.

3. The method according to claim 2, wherein the surface protein is a viral surface protein.

4. A method for the evaluation of the distribution of a cellular receptor for a ligand in an animal comprising the steps of:
a) introducing into a cell of a transgenic non-human animal comprising in its genome one or more viral sequence, but being deficient in one or more viral sequence comprising a gene which encodes a viral surface protein required for the generation of the virus, a viral vector comprising the viral sequence comprising the gene which encodes the viral surface protein, thereby allowing reconstitution of the viral particle generation in the cell of the transgenic animal, wherein the viral sequence encoding the viral surface protein contains the coding sequence of the ligand so that upon expression of this sequence a modified viral surface protein is produced that contains the sequence of the ligand in the part of the protein that is accessible for interaction with the cellular receptor;
b) maintaining the transgenic animal under suitable conditions allowing the production of viral particles in cells of the transgenic animal; and
c) detecting the cells in which viral particles are produced, wherein the production of virus by a cell indicates that the cell contains the cellular receptor.

5. The method according to claim 1, 2 or 4, wherein the transgenic non-human animal is a mammal.

6 The method according to claim 5, wherein the mammal is a rodent.

7. The method according to claim 6, wherein the rodent is a mouse.

8. The method according to claim 7, wherein the mouse is a severe combined immune deficient mouse.

9. The method according to claim 1, 2 or 4, wherein the transgenic animal is deficient in a viral sequence encoding a packaging signal.

10. The method according to claim 9, wherein the viral sequence encoding the packaging signal is deleted.

11. The method according to claim 1, 2 or 4, wherein the expression of the one or more viral sequence comprised in the genome of the transgenic animal is regulated by a ubiquitous, constitutively active regulatory sequence.

12. The method according to claim 1 wherein the regulatory sequence to be analyzed regulates a cellular function selected from the group consisting of expression and translation.

13. The method according to claim 1 wherein the regulatory sequence is operably linked to a surface protein gene.

14. The method according to claim 13, wherein the surface protein gene encodes env.

15. The method according to claim 14, wherein the env is amphotropic.

16. The method according to claim 1, wherein the regulatory sequence to be analyzed is suitable for targeted gene therapy.

17. The method according to claim 2 or 4, wherein the transgenic animal is deficient in a surface protein sequence required for generation of infectious virus because the viral sequence encoding the surface protein has been deleted.

18. The method according to claim 2, wherein the surface protein is suitable for targeted gene therapy.

19. The method of claim 1, 2 or 4 wherein wherein the viral sequences are of retroviral origin.

20. The method according to claim 19, wherein at least one retroviral sequence is introduced via a retroviral vector.

21. The method according to claim 20, wherein at least one retroviral sequence is introduced into the non-human transgenic animal by transduction with a retroviral vector.

22. The method according to claim 20, wherein at least one retroviral sequence is introduced into the non-human transgenic animal by infection with a retroviral particle comprising a retroviral vector.

23. The method according to claim 19, wherein the retroviral sequence introduced and the retroviral sequence comprised in the transgenic animal which together reconstitute viral particle generation are derived from the same type of retrovirus.

24. The method according to claim 1, wherein a viral sequence under control of the regulatory sequence to be analyzed is comprised in a retroviral vector and the regulatory sequence is inserted into a Long Terminal Repeat of the retroviral vector.

25. The method according to claim 24, wherein the regulatory sequence is inserted into a U3-region of the Long Terminal Repeat.

26. The method according to claim 20, wherein the retroviral vector is based on a promoter conversion vector.

27. The method according to claim 20, wherein the retroviral vector further comprises a heterologous gene.

28. The method according to claim 27, wherein the heterologous gene is selected from the group consisting of a therapeutic gene, an anti-tumor gene, a marker gene, green fluorescent protein, and a zeocine resistance gene.

29. The method according to claim 27, wherein the heterologous gene is operatively linked to an internal ribosome entry site.

30. A transgenic animal produced by the method of claim 1, 2 or 4.

31. A transgenic non-human animal comprising in its genome one or more viral sequence, but being deficient in at least one viral sequence required for generation of the virus, wherein an introduction of the viral sequence required for generation of the virus into a cell of the transgenic animal allows reconstitution of viral particle generation and dissemination of said viral particles in the transgenic animal.

32. The transgenic animal according to claim 31, wherein the non-human transgenic animal is a mouse.

33. The transgenic mouse of claim 32 which is a severe combined immunodeficient mouse.

34. The transgenic non-human animal of claim 31 which is deficient in a viral sequence encoding a viral surface protein.

35. The transgenic non-human animal of claim 31, wherein the viral sequences comprised in the animal genome are regulated by a ubiquitous, constitutively active regulatory sequence.

36. The transgenic animal of claim 31, where the viral sequences are of retroviral origin.

37. The transgenic animal of claim 35, wherein the regulatory sequence is selected from the group consisting of the SV40 enhancer/promoter, the beta actin promoter, the ROSA26 promoter, the CDC10 promoter, the ubiquitin promoter and the Murine Leukemia Virus promoter.

38. The transgenic animal according to claim 36, wherein the retroviral sequences are derived from Murine Leukemia Virus.


SEQUENCE LISTING (6 sequences)

SEQ ID NO: 1 - DNA, 20 bp, Artificial Sequence
  Description of Artificial Sequence Primer
gcctcggcctctgagctatt

SEQ ID NO: 2 - DNA, 27 bp, Artificial Sequence
  Description of Artificial Sequence Primer
atatccgcgggctagcttgccaaacct

SEQ ID NO: 3 - DNA, 21 bp, Artificial Sequence
  Description of Artificial Sequence Primer
ctgtgataaactaccgcatta

SEQ ID NO: 4 - DNA, 21 bp, Artificial Sequence
  Description of Artificial Sequence Primer
gtttattgcagcttataatgg

SEQ ID NO: 5 - DNA, 20 bp, Artificial Sequence
  Description of Artificial Sequence Primer
tgcgctgctgagaagccagt

SEQ ID NO: 6 - DNA, 20 bp, Artificial Sequence
  Description of Artificial Sequence Primer
ttgtgagcgatccgctcgac